(19) United States
(12) Patent Application Publication
(10) Pub. No.: US 2023/0293320 A1
(43) Pub. Date: Sep. 21, 2023

(54) METHOD FOR CONTROLLING A PROSTHESIS OR ORTHOSIS

(71) Applicant: Otto Bock Healthcare Products GmbH, Vienna (AT)

(72) Inventors: Martin Seyr, Vienna (AT); Thomas Hofmann, Vienna (AT); Dirk Seifert, Vienna (AT); Philipp Kampas, Vienna (AT); Dries Glorieux, Lommel (BE)

(21) Appl. No.: 18/016,838

(22) PCT Filed: Jul. 20, 2021
(86) PCT No.: PCT/EP2021/070243

§ 371 Date: Jan. 18, 2023

(30) Foreign Application Priority Data
Jul. 20, 2020 (DE) 10 2020 004 336.4

(51) Int. Cl. A61F 2/70; A61F 2/64; A61F 5/01

(52) U.S. Cl. CPC A61F 2/70; A61F 2/64; A61F 5/0125; A61F 2002/7635

(57) ABSTRACT

The invention relates to a method for controlling a prosthesis or orthosis of the lower extremity, which prosthesis or orthosis has an upper part (10) and a lower part (20), which lower part is connected to the upper part (10) by means of a knee joint (1) and is mounted for pivoting relative to the upper part (10) about a joint shaft (15); wherein an adjustable resistance device (40) is disposed between the upper part (10) and the lower part (20), by means of which resistance device a flexion resistance (Rf) is changed on the basis of sensor data; wherein an axial force (FA) acting on the lower part is sensed by at least one sensor (54) and is used as the basis for a change of the flexion resistance (Rf); wherein, in the case of decreasing axial force (FA) and/or an approximately vertical position of a leg tendon (70) and/or of an extended knee joint (1), the flexion resistance (Rf) is reduced; and wherein the flexion resistance (Rf) is increased again if, within a temporally defined interval, no knee flexion is detected and/or the knee joint (1) and/or the leg tendon (70) and/or the axial force (FA) fall below or exceed specific limit values.

The invention relates to a method for controlling a prosthesis or orthosis of the lower extremity, having an upper part and having a lower part which is connected to the upper part via a knee joint and is mounted so as to be pivotable relative to the upper part about a joint axis, wherein there is arranged between the upper part and the lower part an adjustable resistance device by means of which a flexion resistance is changed on the basis of sensor data, wherein an axial force acting on the lower part is detected by at least one sensor and used as the basis for a change of the flexion resistance.

Artificial knee joints are used in prostheses and orthoses as well as in exoskeletons as a special case of orthoses. An artificial knee joint has an upper part and a lower part which are mounted so as to be pivotable relative to one another about a joint axis, the knee axis. In the simplest case, the knee joint is in the form of a single-axis knee joint, in which, for example, a pin or two bearing points arranged on a pivot axis form a single knee axis. Also known are artificial knee joints which do not form a fixed axis of rotation between the upper part and the lower part, but have either sliding or rolling surfaces or a plurality of link bars connected together in an articulated manner. In order to be able to influence the movement properties of the knee joints and obtain a movement behavior of the orthosis or prosthesis, or of the exoskeleton, that emulates natural gait behavior, there are provided between the upper part and the lower part resistance devices by means of which the resistance can be changed. Purely passive resistance devices are passive dampers, for example hydraulic dampers, pneumatic dampers, or dampers that change the movement resistance on the basis of magnetorheological effects. There are also active resistance devices, for example motors or other drives, which, via a corresponding connection, can be operated as generators or energy stores.

The knee joints, that is to say the prosthetic joints or orthotic knee joints, are fixed to the patient by attachment means. In the case of prosthetic knee joints, fixing generally takes place by means of a thigh socket, which receives a limb stump. Alternative types of fixing are likewise possible, for example by osseointegrated attachment means or by means of belts and other devices. In the case of orthoses and exoskeletons, the upper part and lower part are fixed directly to the thigh and the lower leg. The fastening devices provided for that purpose are, for example, belts, sleeves, cups or frame structures. Orthoses can also have foot parts for supporting a foot or shoe. The foot parts can be mounted in an articulated manner on the lower part.

DE 10 2013 011 080 A1 relates to a method for controlling an orthopedic joint device of a lower extremity, having an upper part and a lower part mounted in an articulated manner thereon, between which there is arranged a conversion device by means of which mechanical work from the relative movement during a pivoting of the upper part relative to the lower part is converted and stored at least in one energy store. The stored energy is re-supplied to the joint device in a time-delayed manner in order to assist the pivoting of the upper part and lower part in the course of the movement. Assistance of the relative movement takes place in a controlled manner. In addition to the conversion device there can be provided a separate damper which is in the form of a hydraulic damper or pneumatic damper and is adjustable, so that, by means of the damper device, the resistance can be influenced both in the flexion direction and in the extension direction during walking.

An artificial knee joint with the maximum extension that is constructionally achievable has a knee angle of 180°, a hyperextension, which corresponds to an angle on the posterior side of more than 180°, is generally not provided. Pivoting of the lower part posteriorly relative to the upper part is referred to as knee flexion, pivoting anteriorly or in a forward direction is referred to as extension.

DE 10 2006 021 802 A1 discloses control of a passive prosthetic knee joint with adjustable damping of the flexion resistance. The adaptation is made to climbing stairs, wherein a low-moment lifting of the prosthetic foot is detected and the flexion damping, in the lifting phase, is lowered to below a level suitable for walking on a level surface. The flexion damping can be increased in dependence on the change of the knee angle and in dependence on the axial force acting on the lower leg.

For alternating walking on a level surface, there are additionally control methods which allow the flexion resistance to be adapted in dependence on the particular gait situation. Special situations which make inflexion of the knee joint necessary, for example moving off from a standing position, in particular with the prosthesis or orthosis first, are problematic.

The object of the present invention is, therefore, to provide a method with which artificial knee joints can be used more comfortably for a user.

According to the invention, said object is achieved by means of a method having the features of the main claim. Advantageous embodiments and developments of the invention are disclosed in the dependent claims, in the description and in the figures.

The method for controlling a prosthesis or orthosis of the lower extremity, having an upper part and having a lower part which is connected to the upper part via a knee joint and is mounted so as to be pivotable relative to the upper part about a joint axis, wherein there is arranged between the upper part and the lower part an adjustable resistance device by means of which a flexion resistance is changed on the basis of sensor data, wherein an axial force acting on the lower part is detected by at least one sensor and used as the basis for a change of the flexion resistance, provides that, in the case of a decreasing axial force and/or an approximately vertical position of a leg cord and/or an extended knee joint, the flexion resistance is reduced, wherein the flexion resistance is raised again if, within a fixed period of time, no knee flexion is detected and/or the knee joint and/or the leg cord and/or the axial force exceed specific limit values. The above-mentioned conditions are no longer met, for example, if the knee joint is largely or completely relieved of load in the axial direction. The axial force is detected, for example, by an axial force sensor which is arranged on the prosthesis or orthosis, in particular on the lower part a component fastened to the lower part. If a forward rotation of the joint axis is detected, which can occur, for example, during a rolling movement over the foot or as a result of a flexion of the lower leg about an ankle joint axis, the flexion resistance is reduced. The forward rotation of the joint axis and thus also of the knee joint as a whole means that the joint axis and thus also a proximal end of the lower part is pivoted about a distal rotation point, wherein the distal rotation point can be either a joint axis at an ankle joint or a moving point in the region of the sole of the foot. The flexion resistance is likewise reduced if, alternatively or in addition, a vertical position of a leg cord is detected. The leg cord is in particular defined as a connecting line between two defined points on the upper part and on the lower part or a component attached to the lower part. A preferred embodiment provides that the leg cord is defined as the connecting line between a point that is spaced apart proximally from the joint axis on the upper part and a point that is spaced apart distally from the joint axis on the lower part, for example a hip rotation point and a foot point. In the case of the use of a prosthetic knee joint, the hip rotation point is in any case determined by an orthopedic technician and defines the segment length of the thigh or upper part, which is defined as the distance between the joint axis or knee axis and the hip rotation point. The segment length of the lower part is defined by the distance between the knee axis and a foot point. There can be defined as the foot point, for example, the middle of the foot, the instantaneous center of a rolling movement, the end point of the perpendicular of the lower leg at the level of the sole of the foot part, of the prosthetic foot or on the ground, other points close to the ground are likewise suitable for defining a foot point. Because a foot part for supporting a natural foot that is still present is not necessary in the case of orthoses or exoskeletons, the distance from the ground to the joint axis can also be used. The position and/or the length of the leg cord provide reliable information about the orientation of the leg and the movement progression. The leg cord can be calculated or assessed by means of absolute angle sensors in conjunction with the known segment lengths, an absolute angle sensor and a knee angle sensor. A positive leg cord angle is present if the leg cord is tilted in the posterior direction in the sagittal plane. This is the case if, for example, the foot or the ankle joint axis is located in front of the knee or the knee joint axis when seen in the forward walking direction. A negative leg cord angle is present if the leg cord is tilted forward, for example when the knee joint and the hip joint are located in front of the knee joint axis. In the case of a positive leg cord angle, an increasing distance of the leg cord from the vertical is numbered positively as an increase or enlargement. In the case of a negative leg cord angle, an increasing distance of the leg cord from the vertical is numbered negatively as a decrease or reduction.

The flexion resistance is likewise reduced if, alternatively or in addition, an extended knee joint is detected. The reduction of the flexion resistance is maintained only for a fixed period of time and is reversed again, wherein the flexion resistance can be raised to the same flexion resistance level or to a different flexion resistance level. The flexion resistance is raised in particular if no knee flexion is detected within the fixed period of time.

Alternatively or in addition, the flexion resistance is raised if the knee joint and/or the leg cord are no longer in an approximately vertical position and/or the knee joint is no longer largely or completely relieved of load in the axial direction. Even when the knee joint is completely relieved of load, if a ground reaction force no longer acts in the direction of the knee joint with the lower part extended longitudinally, the flexion resistance is raised if at least one of the other criteria is met, in order to raise the flexion resistance again. With the method, it is possible to achieve easier inflexion of the knee joint, for example for setting off from a standing position. In the case where the prosthesis or orthosis is relieved of load, for example if the body weight is displaced to the contralateral side, the flexion resistance is reduced automatically in order to be able to carry out flexion without flexion resistance and thus a significantly reduced flexion resistance. It is thus possible to bring the knee joint forward slightly despite contact with the ground, without the orthosis or prosthesis having to be lifted from the ground completely by a compensating movement in the hip and the pelvis. The foot or prosthetic foot is able to roll forward until, as a result of the flexion in the knee joint, the effective length of the prosthesis or orthosis is reduced to such an extent that a forward swing without ground contact is made possible. By means of the method, the knee joint remains secured in the stance phase in the case of an axial load, whereby the patient acquires increased stability and great confidence in the prosthesis or orthosis. At the same time, sufficient dynamics is provided within the knee joint, which allows a swing phase to be initiated sufficiently comfortably even in special situations.

A further development of the invention provides that the flexion resistance is reduced when setting off from a standing position, in particular is reduced only when setting off from a standing position. The situation of standing can be recognized or detected, for example, by detection of the axial force over time. If the axial force remains the same or approximately the same for a defined period of time, it can be assumed that the user of the prosthesis or orthosis is not moving but is stationary. When standing on both legs, users of orthoses or prostheses usually stand with approximately half their body weight on the prosthesis or orthosis, possibly with slightly less weight. This weight range can be specified as the limit value. If the measured axial force is in this limit value range over a specific time period, this can be regarded as a condition for initiating the above-described method. The same can take place by monitoring a flexion angle. If the knee joint is not flexed and is in an extended position for a specific period of time, this can serve, on its own or in conjunction with the axial force monitoring, as an indication that the user of the orthosis or prosthesis is standing upright. Standing can be distinguished from a movement or walking and/or a gait cycle by one or more IMUs.

The flexion resistance can be reduced in dependence on the decrease in the axial force, in particular a degressive reduction of the flexion resistance toward a target value is advantageous. In the case of an initially small reduction in the axial force, a comparatively great reduction in the flexion resistance occurs, so that, starting from, for example, locking as a result of a maximum hydraulic resistance, in the case of a comparatively small decrease in the axial force inflexion against a flexion resistance is in principle possible. As the axial force decreases further, the reduction will take place in a less pronounced manner.

The flexion resistance can be reduced to a level below a stance phase damping, in particular to the level below a stance phase damping when walking on a level surface.

The flexion resistance can be reduced in dependence on the axial force, the leg cord angle and/or the spatial angle of the lower part, wherein a plurality or all of the parameters can be taken into consideration when calculating and determining how the reduction of the flexion resistance should take place. In addition to a pure change-over of the flexion resistance when fixed limit values are reached or exceeded or fallen below, smooth transitions and resistance changes can be set and brought about in dependence on changing parameters.

In a development of the invention, in the case of a decrease of the axial force, starting from a starting value, for example an axial load while standing in an unloaded state on both legs, to a level above a limit value, for example to above 10% of the body weight, and a determined leg cord angle above a limit value, in particular above 5°, no reduction of the flexion resistance takes place. If the leg cord is displaced backward, for example pivots backward or posteriorly by an angle of 5° or more, no reduction of the flexion resistance takes place if a sufficiently large reduction of the axial force has taken place. The hip or the hip joint is here displaced behind the foot or the distal reference point for determining the leg cord. In the case of a sufficiently large decrease in the axial force and a corresponding backward rotation of the leg cord, it can be concluded that a patient wishes to sit down, for which purpose an increased flexion resistance is advantageous in order to provide increased security against collapsing of the knee joint. If a reduced rotation of the leg cord in the backward direction is detected, the flexion resistance is reduced accordingly, where backward rotation of the leg cord is not present, a complete reduction of the flexion resistance is possible.

A variant of the invention provides that, in the case of a decrease of the axial force to a level below a limit value, for example to a level below 10% of the body weight, and a determined leg cord angle outside a defined angle range about the vertical, for example in the case of a positive leg cord angle of more than 30° or in the case of a negative leg cord angle of less than −10°, no reduction of the flexion resistance takes place. Such a situation can occur, for example, when walking backward or when climbing over an obstacle with a large forward step.

A complete reduction of the flexion resistance can take place in the case of a positive leg cord angle of up to 20°, wherein the flexion resistance is increased in the case of a larger leg cord angle. Alternatively, a complete reduction of the flexion resistance can take place from a negative leg cord angle of less than −10°. By contrast, the flexion resistance can be increased in the case of a smaller leg cord angle.

A variant of the invention provides that, in the case of a decrease of the axial force to a level below a limit value, for example below 10% of the body weight of the user of the orthosis or prosthesis, and a determined inclination angle of the lower part within a defined angle range about the vertical, that is to say the so-called roll angle is located in a defined range in the vicinity of the vertical, no reduction of the flexion resistance takes place, in particular if the positive roll angle is less than 15° and the negative roll angle is greater than −5°.

A complete reduction of the flexion resistance can take place in the case of a positive inclination angle of the lower part of 20° or more, wherein the flexion resistance is increased in the case of a smaller inclination angle. Alternatively, it is provided that, from a negative inclination angle of −10°, a complete reduction of the flexion resistance takes place, and the flexion resistance is increased or is not reduced in the case of a larger negative inclination angle, that is to say if the lower part is inclined in the direction of the vertical.

The flexion resistance can be increased if an extension movement takes place in the knee joint, which can be detected by way of a knee angle sensor. This can likewise take place by the evaluation of IMU data. The flexion resistance is likewise increased if a gait cycle is detected, for example by means of recurring load patterns or movement patterns such as regular flexion angles in the knee joint or in the ankle joint. The flexion resistance can likewise be increased in the case of an increase in the axial force.

The flexion resistance can be not reduced if a backward inclination of the lower part is detected. In particular, the method serves to facilitate sitting down, walking backward, climbing over an obstacle, and placing a foot or an orthosis on a next lower stair edge or step when walking downstairs. In the mentioned cases, the reduction of the flexion resistance, which is intended to facilitate setting off from a standing position, is not carried out or not carried out to the same extent or is reversed. Thus, not only is the movement sequence for the mentioned cases facilitated, but sufficient security is also ensured for the user in such cases.

In particular, the method serves for controlling a prosthesis or orthosis of the lower extremity, having an upper part and having a lower part which is connected to the upper part via a knee joint and is mounted so as to be pivotable relative to the upper part about a joint axis, wherein there is arranged between the upper part and the lower part an adjustable resistance device by means of which a flexion resistance is changed on the basis of sensor data, wherein an axial force acting on the lower part is detected by at least one sensor and used as the basis for a change of the flexion resistance, for setting off from a standing position, in which the flexion resistance is reduced from an initial value in the case of a decreasing axial force, in particular if the flexion angle does not exceed a limit value. The limit value can in particular be fixed at a value of not greater than 10°. The method serves in particular for adjusting the flexion resistance if the user is not in a gait cycle and wishes to perform a movement other than walking on a level surface. A damping reduction or a reduction of the resistance does not take place or is discontinued if it is recognized that the user changes to a gait cycle, a knee extension takes place, or the axial load in the direction of the joint axis increases again.

An exemplary embodiment of the invention will be discussed in more detail below on the basis of the appended figures. In the figures:

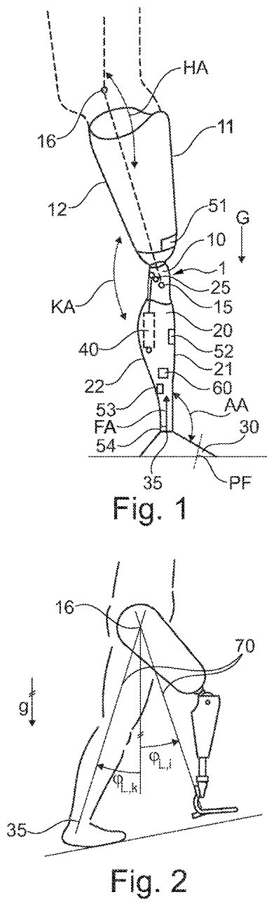

FIG. 1—shows a schematic illustration of a prosthetic leg;

FIG. 2—shows an illustration of leg cords;

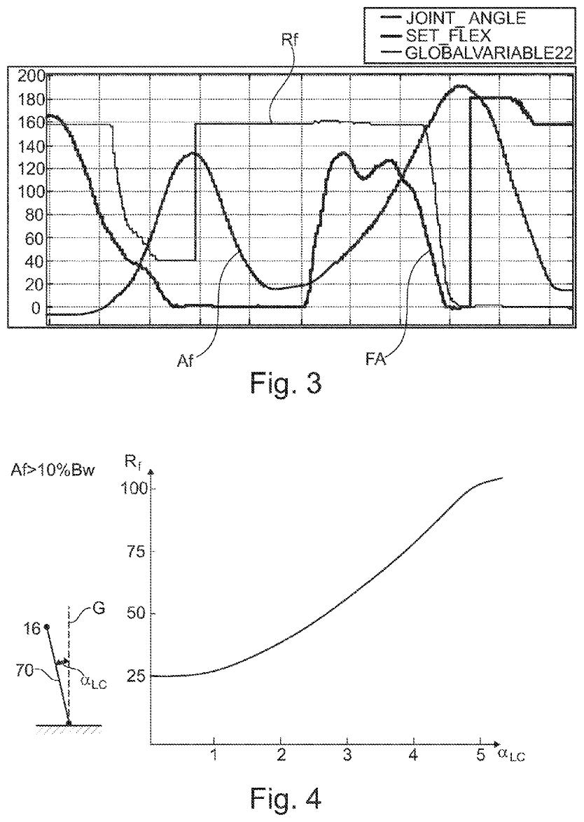

FIG. 3—shows a profile of axial force, resistance and knee angle when walking downstairs;

FIGS. 4 to 6—show flexion resistance profiles over leg cord angles and rolling angles; and

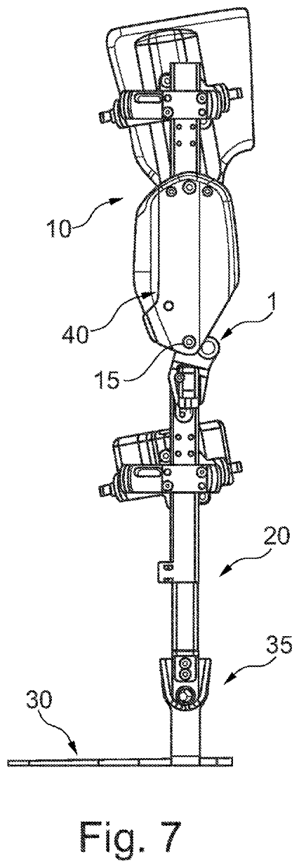

FIG. 7—shows an illustration of an orthosis.

FIG. 1 shows a schematic illustration of an artificial knee joint 1 in an application in a prosthetic leg. As an alternative to an application in a prosthetic leg, a correspondingly designed artificial knee joint 1 can also be used in an orthosis or an exoskeleton. Instead of replacing a natural joint, the artificial knee joint 1 is then arranged medially and/or laterally on the natural joint. In the exemplary embodiment shown, the artificial knee joint 1 is in the form of a prosthetic knee joint having an upper part 10 with a side 11 which is anterior or situated in the walking direction or at the front, and a posterior side 12 which is located opposite the anterior side 11. A lower part 20 is arranged on the upper part 10 so as to be pivotable about a pivot axis 15. The lower part 20 also has an anterior side 21 or front side and a posterior side 22 or rear side. In the exemplary embodiment shown, the knee joint 1 is in the form of a monocentric knee joint, it is in principle also possible to control a polycentric knee joint in a corresponding manner. At the distal end of the lower part 20 there is arranged a foot part 30 which can be connected to the lower part either in the form of a rigid foot part 30 with a fixed foot joint or by a pivot axis 35, in order to make possible a movement sequence which emulates the natural movement sequence.

Between the posterior side 12 of the upper part 10 and the posterior side 22 of the lower part 20, the knee angle KA is measured. The knee angle KA can be measured directly by means of a knee angle sensor 25, which can be arranged in the region of the pivot axis 15. The knee angle sensor 25 can be coupled with a torque sensor or can have such a sensor, in order to detect a knee moment about the joint axis 15. On the upper part 10 there is arranged an inertial angle sensor or an IMU 51, which measures the spatial position of the upper part 10, for example in relation to a constant force direction, for example gravitational force G, which points vertically downward. An inertial angle sensor or an IMU 53 is likewise arranged on the lower part 20 in order to determine the spatial position of the lower part while the prosthetic leg is in use.

In addition to the inertial angle sensor 53, an acceleration sensor and/or transverse force sensor 53 can be arranged on the lower part 20 or on the foot part 30. By means of a force sensor or torque sensor 54 on the lower part 20 or on the foot part 30, an axial force FA acting on the lower part 20 or an ankle moment acting about the ankle joint axis 35 can be determined.

Between the upper part 10 and the lower part 20 there is arranged a resistance device 40 in order to influence a pivoting movement of the lower part 20 relative to the upper part 10. The resistance device 40 can be in the form of a passive damper, in the form of a drive, or in the form of a so-called semi-active actuator with which it is possible to store movement energy and purposively release it again at a later time in order to slow or assist movements. The resistance device 40 can be in the form of a linear or rotary resistance device. The resistance device 40 is connected to a control device 60, for example in a wired manner or via a wireless connection, which in turn is coupled with at least one of the sensors 25, 51, 52, 53, 54. The control device 60 electronically processes the signals transmitted by the sensors, using processors, computing units or computers. It has an electrical power supply and at least one memory unit in which programs and data are stored and in which a working memory for processing data is provided. After processing of the sensor data, an activation or deactivation command with which the resistance device 40 is activated or deactivated is outputted. By activation of an actuator in the resistance device 40 it is possible, for example, to open or close a valve or to generate a magnetic field, in order to change a damping behavior.

To the upper part 10 of the prosthetic knee joint 1 there is fastened a prosthesis socket, which serves to receive a thigh stump. The prosthetic leg is connected via the thigh stump to the hip joint 16, on the anterior side of the upper part 10 a hip angle HA is measured, which is marked on the anterior side 11 between a vertical line through the hip joint 16 and the longitudinal extension of the upper part 10 and the connecting line between the hip joint 16 and the knee joint axis 15. If the thigh stump is lifted and the hip joint 16 is flexed, the hip angle HA decreases, for example when sitting down. Conversely, the hip angle HA increases in the case of an extension, for example when standing up or in the case of similar movement sequences.

During a gait cycle when walking on a level surface, the foot part 30 is placed down heel first, the first contact of the heel or of a heel part of the foot part 30 is called heel strike. A plantar flexion then takes place until the foot part 30 rests completely on the ground, the longitudinal extension of the lower part 10 is here generally behind the vertical, which runs through the ankle joint axis 35. When walking on a level surface, the center of mass is then displaced forward, the lower part 20 pivots forward, the ankle angle AA becomes smaller, and there is an increasing load on the forefoot. The ground reaction force vector moves forward from the heel to the forefoot. At the end of the stance phase, a toe-off takes place, which is followed by the swing phase, in which the foot part 30, when walking on a level surface, is displaced behind the center of mass or the hip joint on the ipsilateral side, with a reduction of the knee angle KA, in order then, after a minimum knee angle KA has been reached, to be rotated forward in order then, with a knee joint 1 that is generally extended to the maximum, to achieve heel contact again. The force transmission point PF thus moves during the stance phase from the heel to the forefoot and is illustrated schematically in FIG. 1.

In FIG. 2, a definition of the leg cords 70 of an ipsilateral, assisted leg and of a contralateral, unassisted leg is given. The leg cord passes through the hip rotation point 16 and forms a line to the ankle joint 35. As can be seen in FIG. 2, the length of the leg cord and the orientation φ_{L} of the leg cords 70 changes during the movement, in particular also in the case of different gradients. The profile of the change of the length and/or orientation of the leg cords 70 can be used to assess and predict or determine height differences ΔH that are to be overcome. The respective control commands are then derived therefrom. The orientation of the ipsilateral leg cord φ_{Li} relative to the direction of gravity G and the contralateral leg cord φ_{Lk} is plotted in each case.

FIG. 3 shows the change of the flexion resistance Rf together with the profile of the flexion angle Af and the axial force profile FA. The gait situation corresponds to setting off with the prosthesis side at the beginning of a staircase, with the prosthesis being placed on the next lower step and a knee flexion without reduced flexion resistance. At the beginning of the movement, at the left-hand end of the flexion angle profile, the knee joint is extended to the maximum, the knee angle KA is approximately 180°, the flexion angle Af is thus 00 or approximately 0. The prosthetic knee joint is loaded to the maximum with an axial force FA, and the user of the prosthesis wishes to begin with the assisted leg or the ipsilateral leg and walk downstairs. For this purpose, the axial force FA is first reduced, the flexion resistance Rf is also reduced with a slight time delay, so that inflexion is facilitated and an increase in the flexion angle Af can take place. The flexion resistance Rf is reduced to approximately 25% of the initial value. A complete elimination of damping or of the flexion resistance Rf is not provided. Even if the prosthetic knee joint is relieved of load completely, no further decrease in the flexion resistance Rf takes place if the axial load FA is eliminated. The knee joint flexes, the flexion angle Af increases, so that the knee joint and the joint axis can be brought forward by a flexion of the hip joint. The foot or the prosthetic foot pivots beyond the edge of the step, so that there is an extension movement and thus a reversal of movement of the profile of the flexion angle Af. When a maximum flexion angle has been reached and there has been a reversal of movement, the flexion resistance Rf is very quickly increased to the initial value again and remains at the starting level.

As the movement continues, until the prosthetic foot is in contact with the next lower step, which can be recognized by a pronounced increase in the axial force FA, the flexion resistance Rf remains at the high level so that secure stance phase damping is ensured after the assisted leg has been placed down. The flexion resistance Rf is reduced again only after the axial force FA has fallen, that is to say when the prosthetic knee joint is relieved of load again for the purpose of walking on a level surface or for walking downstairs further.

FIG. 4 shows the profile of a change of the resistance Rf in dependence on the axial force Af and the leg cord angle α_{LC}. A positive leg cord angle α_{LC} of a leg cord is present when the distal reference point or foot point is taken as the starting point and the leg cord 70 is tilted in the posterior direction relative to the vertical or line of gravity G. A schematic illustration of the orientation is shown in the left-hand part of FIG. 4. The further the leg cord 70 is tilted backward, that is to say the hip joint 16 is located behind the foot point or the ankle joint in the sagittal plane, the greater the positive inclination angle of the leg cord 70. In the case of a reduced axial loading of the prosthetic leg to, for example, a force that corresponds to more than 10% of the total body weight, for example between 40% and 15% of the body weight, the resistance Rf is reduced to the maximum extent in the case of an almost vertical orientation, in the exemplary embodiment shown to 25% of the initial resistance. In the case of an increasing backward inclination of the leg cord 70, in the case of an increase in the leg cord angle α_{LC} in the positive direction, the flexion resistance Rf is reduced less until, at a limit value, which in the exemplary embodiment shown is fixed at a backward inclination of 5°, no reduction of the flexion resistance Rf is carried out and the flexion resistance Rf is 100%.

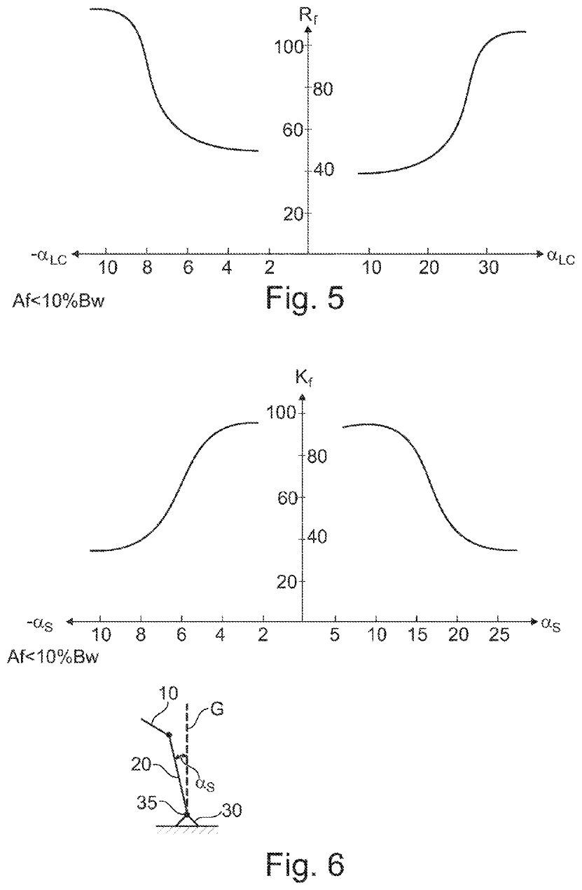

FIG. 5 shows a further variant of the reduction of the flexion resistance Rf in dependence on the axial loading and the leg cord angle α_{LC}. In the case of axial loading with less than 10% of the body weight, for example between 0% and 10% of the body weight, that is to say in the case of a further axial load reduction compared to standing on two legs without a load, the flexion damping or the flexion resistance Rf is adjusted differently than in the case of a small relief of load as in FIG. 4. In the case of a very considerable backward inclination of the leg cord 70 at an angle of between 20° and 30°, for example when climbing over an obstacle, no or only a limited reduction of the flexion resistance Rf is carried out. The increase takes place from a leg cord angle α_{LC} of 20°, until then a reduction of the resistance to the target value can take place in the case of an axial force reduction. No reduction takes place from an angle of 30°. In the case of a negative leg cord orientation, that is to say in the case of a forward displacement of the leg cord 70, a reduction to the target value, in the exemplary embodiment shown to 40% of the maximum resistance, will only take place from 10°, in the case of a greater forward inclination a lesser reduction or no reduction at all is allowed, even if an axial load reduction occurs. A negative leg cord angle α_{LC} is found, for example, when walking backwards. The lowering and raising of the flexion resistance Rf can be carried out, as shown in FIG. 5, over a particular angle range, alternatively the transition can also take place in the form of a sudden lowering and raising. Such a type of adjustment has been found to be advantageous in particular in the negative angle range, that is to say in the case of a forward inclination of the lower part 20.

FIG. 6 shows a further example of the dependence of the resistance reduction on further sensor signals according to the loading state. The axial force Af is reduced not to a level according to FIG. 4, but to a level according to FIG. 5, so that the reduced axial force Af is not more than 10% of the body weight. The axial force can be reduced, for example, to 0% or 5% of the body weight on the assisted leg. FIG. 6 shows, as a further criterion for reducing the flexion resistance, the roll angle α_{S}, which is measured between the lower part 2 and the vertical G. The vertical G runs through the pivot axis 35 of the ankle joint between the foot part 30 and the lower part 20 or through the rotation point at ground level if the foot part 30 is rigidly coupled with the lower part 20. A displacement in the posterior direction is a positive roll angle α_{S}. In the case of a displacement forward, so that the knee joint lies with the joint axis 15 in front of the vertical G, a negative roll angle α_{S} is present. If, for example, the negative roll angle is more than minus 10° relative to the vertical, the flexion resistance Rf is reduced completely, here too to the level of 40% of the initial resistance. In the case of a smaller forward inclination, that is to say in the case of a smaller negative roll angle α_{S}, the flexion resistance Rf remains greater, the reduction thus becomes smaller. In the case of a positive roll angle α_{S}, a complete reduction to the target value of the flexion resistance Rf takes place from an angle of 20°, no reduction takes place up to an angle of 15°.

FIG. 7 shows, in a schematic illustration, an exemplary embodiment of an orthosis having an upper part 10 and a lower part 20 mounted thereon so as to be pivotable about a pivot axis 15, with which the method can likewise be carried out. Between the upper part 10 and the lower part 20 there is formed an artificial knee joint 1, which in the exemplary embodiment shown is arranged laterally to a natural knee joint. In addition to an arrangement of the upper part 10 and lower part 20 on one side relative to a leg, it is also possible for two upper parts and lower parts to be arranged medially and laterally to a natural leg. The lower part 20 has at its distal end a foot part 30 which is mounted so as to be pivotable relative to the lower part 20 about an ankle joint axis 35. The foot part 30 has a foot plate on which a foot or shoe can be supported. Both on the lower part 20 and on the upper part 30 there are arranged fastening devices for fixing to the lower leg or the thigh. Devices for fixing the foot on the foot part 30 can also be arranged on the foot part 30. The fastening devices can be in the form of buckles, belts, clips or the like, in order to allow the orthosis to be releasably placed on the leg of the user and removed again without being damaged. To the upper part 10 there is fastened the resistance device 40, which bears against the upper part 20 and against the lower part 10 and provides an adjustable resistance to pivoting about the pivot axis 15. The sensors and the control device described above in connection with the exemplary embodiment of the prosthesis are correspondingly present also on the orthosis.

1. A method for controlling a prosthesis or orthosis of the lower extremity, having an upper part (10) and having a lower part (20) which is connected to the upper part (10) via a knee joint (1) and is mounted so as to be pivotable relative to the upper part (10) about a joint axis (15), wherein there is arranged between the upper part (10) and the lower part (20) an adjustable resistance device (40) by means of which a flexion resistance (Rf) is changed on the basis of sensor data, wherein an axial force (AF) acting on the lower part is detected by at least one sensor (54) and used as the basis for a change of the flexion resistance (Rf), characterized in that
a. in the case of a decreasing axial force (FA) and/or an approximately vertical position of a leg cord (70) and/or an extended knee joint (1), the flexion resistance (Rf) is reduced,
b. wherein the flexion resistance (Rf) is raised again if, within a fixed period of time, no knee flexion is detected and/or the knee joint (1) and/or the leg cord (70) and/or the axial force (FA) exceed or fall below specific limit values.

2. The method as claimed in claim 1, characterized in that the flexion resistance (Rf) is reduced when setting off from a standing position.

3. The method as claimed in claim 1, characterized in that the flexion resistance (Rf) is reduced in dependence on the decrease in the axial force (FA).

4. The method as claimed in claim 1, characterized in that the flexion resistance (Rf) is reduced to a level below a stance phase resistance.

5. The method as claimed in claim 1, characterized in that the flexion resistance (Rf) is reduced in dependence on the axial force (FA), the leg cord angle (α_{LC}) and/or a spatial angle (α_{S}) of the lower part (20).

6. The method as claimed in claim 1, characterized in that, in the case of a decrease of the axial force (FA) to a level above a limit value and a determined positive leg cord angle (α_{LC}) above a limit value, in particular above 5°, no reduction of the flexion resistance (Rf) takes place.

7. The method as claimed in claim 1, characterized in that, in the case of a decrease of the axial force (FA) to a level below a limit value and a determined leg cord angle (α_{LC}) outside a defined angle range about the vertical (G), in particular in the case of a positive leg cord angle (α_{LC}) greater than 30° and a negative leg cord angle (α_{LC}) of less than −10°, no reduction of the flexion resistance (Rf) takes place.

8. The method as claimed in claim 7, characterized in that a complete reduction of the flexion resistance (Rf) takes place in the case of a positive leg cord angle (α_{LC}) of up to 20° and the flexion resistance (Rf) is increased in the case of a larger leg cord angle (α_{LC}), or in that a complete reduction of the flexion resistance (Rf) takes place from a negative leg cord angle (α_{LC}) of −10° and the flexion resistance (Rf) is increased in the case of a smaller leg cord angle (α_{LC}).

9. The method as claimed in claim 1, characterized in that, in the case of a decrease of the axial force (FA) to a level below a limit value, in particular below 10% of the body weight of the patient, and a determined inclination angle (α_{S}) of the lower part (20) relative to the vertical (G) within a defined angle range about the vertical (G), in particular within a range between a positive inclination angle (α_{S}) of less than 15° and a negative inclination angle (α_{S}) of greater than −5°, no reduction of the flexion resistance (Rf) takes place.

10. The method as claimed in claim 1, characterized in that a complete reduction of the flexion resistance (Rf) takes place in the case of a positive inclination angle (α_{S}) of the lower part (20) of 20° or more and the flexion resistance (Rf) is increased in the case of a smaller inclination angle (α_{S}), or in that a complete reduction of the flexion resistance (Rf) takes place from a negative inclination angle (α_{S}) of the lower part (20) of −10° and the flexion resistance (Rf) is increased in the case of a larger negative inclination angle (α_{S}).

11. The method as claimed in claim 1, characterized in that the flexion resistance (Rf) is increased if an extension movement takes place, a gait cycle is detected and/or an increase of the axial force (FA) is detected.

12. The method as claimed in claim 1, characterized in that the flexion resistance is not reduced if a backward inclination of the lower part (20) is detected.
